SEQUENCE LISTING (44 sequences)

SEQ ID NO: 1 - protein, 150 aa, Homo sapiens
MGSSHHHHHHSSGLVPRGSHMPASVQLHTAVEMHHWCIPFSVDGQPAPSLRWLFNGSVLNETSFIFTEFLEPAANETVRHGCLRLNQPTHVNNGNYTLLAANPFGQASASIMAAFMDNPFEFNPEDPIPVSFSPVDTNSTSGDPVEKKDE

SEQ ID NO: 2 - protein, 119 aa, Homo sapiens
MGSSHHHHHHSSGLVPRGSHMAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPG

SEQ ID NO: 3 - protein, 166 aa, Homo sapiens
MGSSHHHHHHSSGLVPRGSHMAPTITFLESPTSDHHWCIPFTVKGNPKPALQWFYNGAILNESKYICTKIHVTNHTEYHGCLQLDNPTHMNNGDYTLIAKNEYGKDEKQISAHFMGWPGIDDGANPNYPDVIYEDYGTAANDIGDTTNRSNEIPSTDVTDKTGREH

SEQ ID NO: 4 - protein, 149 aa, Homo sapiens
MGSSHHHHHHSSGLVPRGSHMTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEPFPESTDNFILFDEVSPTPPITVTHKPEE

SEQ ID NO: 5 - protein, 121 aa, Homo sapiens
MGSSHHHHHHSSGLVPRGSHMTVYYPPRVVSLEEPELRLEHCIEFVVRGNPPPTLHWLHNGQPLRESKIIHVEYYQEGEISEGCLLFNKPTHYNNGNYTLIAKNPLGTANQTINGHFLKEP

SEQ ID NO: 6 - DNA, 36 bp, Artificial Sequence
  TrkBIg2 6His forward primer
cgcatatggcaccaactatcacatttctcgaatctc

SEQ ID NO: 7 - DNA, 35 bp, Artificial Sequence
  TrkBIg2 6His Reverse Primer
gcggatccctattaatgrrcccgaccggttttatc

SEQ ID NO: 8 - DNA, 27 bp, Artificial Sequence
  TrkCIg2 6His Forward Primer
cgcatatgactgtctactatcccccac

SEQ ID NO: 9 - DNA, 34 bp, Artificial Sequence
  TrkCIg2 6His Reverse Primer
gcggatccttatcagggctccttgaggaagtggc

SEQ ID NO: 10 - DNA, 43 bp, Artificial Sequence
  TrkAIg2 noHis Forward Primer
ggaattccatatgcctgcttcagtacaattacacacggcggtc

SEQ ID NO: 11 - DNA, 43 bp, Artificial Sequence
  TrkAIg2 noHis Reverse Primer
ccgctcgagttatcattcgtccttcttctccaccgggtctcca

SEQ ID NO: 12 - DNA, 30 bp, Homo sapiens
atgccggccagtgtgcagctgcacacggcg

SEQ ID NO: 13 - DNA, 30 bp, Artificial Sequence
  TrkAIg2 mut7
atgcctgcttcagtacaattacacacggcg

SEQ ID NO: 14 - protein, 10 aa, Homo sapiens
MPASVQLHTA

SEQ ID NO: 15 - DNA, 30 bp, Homo sapiens
atggcaccaactatcacatttctcgaatct

SEQ ID NO: 16 - DNA, 30 bp, Artificial Sequence
  TrkBIg2 mut1
atggcgccgaccattaccttcctcgaatct

SEQ ID NO: 17 - DNA, 30 bp, Artificial Sequence
  TrkBIg2 mut2
atggcgccaacaataactttcctcgaatct

SEQ ID NO: 18 - DNA, 30 bp, Artificial Sequence
  TrkBIg2 mut3
atggcgcctacgattacgttcctcgaatct

SEQ ID NO: 19 - DNA, 30 bp, Artificial Sequence
  TrkBIg2 mut4
atggccccgaccattaccttcctcgaatct

SEQ ID NO: 20 - DNA, 30 bp, Artificial Sequence
  TrkBIg2 mut5
atggccccaacaataactttcctcgaatct

SEQ ID NO: 21 - DNA, 30 bp, Artificial Sequence
  TrkBIg2 mut6
atggcccctacgattacgttcctcgaatct

SEQ ID NO: 22 - DNA, 30 bp, Artificial Sequence
  TrkBIg2 mut7
atggctccgaccattaccttcctcgaatct

SEQ ID NO: 23 - DNA, 30 bp, Artificial Sequence
  TrkBIg2 mut8
atggctccaacaataactttcctcgaatct

SEQ ID NO: 24 - DNA, 30 bp, Artificial Sequence
  TrkBIg2 mut9
atggctcctacgattacgttcctcgaatct

SEQ ID NO: 25 - protein, 10 aa, Homo sapiens
MAPTITFLES

SEQ ID NO: 26 - DNA, 30 bp, Homo sapiens
atgactgtctactatcccccacgtgtggtg

SEQ ID NO: 27 - DNA, 30 bp, Artificial Sequence
  TrkCIg2 mut1
atgacggtgtattaccctccacgtgtggtg

SEQ ID NO: 28 - DNA, 30 bp, Artificial Sequence
  TrkCIg2 mut2
atgacggtatattatccaccgcgtgtggtg

SEQ ID NO: 29 - DNA, 30 bp, Artificial Sequence
  TrkCIg2 mut3
atgacggtttactacccgcctcgtgtggtg

SEQ ID NO: 30 - DNA, 30 bp, Artificial Sequence
  TrkCIg2 mut4
atgaccgtgtattaccctccacgtgtggtg

SEQ ID NO: 31 - DNA, 30 bp, Artificial Sequence
  TrkCIg2 mut5
atgaccgtatattatccaccgcgtgtggtg

SEQ ID NO: 32 - DNA, 30 bp, Artificial Sequence
  TrkCIg2 mut6
atgaccgtttactacccgcctcgtgtggtg

SEQ ID NO: 33 - DNA, 30 bp, Artificial Sequence
  TrkCIg2 mut7
atgacagtgtattaccctccacgtgtggtg

SEQ ID NO: 34 - DNA, 30 bp, Artificial Sequence
  TrkCIg2 mut8
atgacagtatattatccaccgcgtgtggtg

SEQ ID NO: 35 - DNA, 30 bp, Artificial Sequence
  TrkCIg2 mut9
atgacagtttactacccgcctcgtgtggtg

SEQ ID NO: 36 - protein, 10 aa, Homo sapiens
MTVYYPPRVV

SEQ ID NO: 37 - protein, 21 aa, Artificial Sequence
  pET15b peptide sequence
MGSSHHHHHHSSGLVPRGSHM

SEQ ID NO: 38 - DNA, 54 bp, Artificial Sequence
  TrkAIg2 6His sequence
gaaggagatataccatgggcagcagccatcatcatcatcatcacagcagcggcc

SEQ ID NO: 39 - DNA, 13 bp, Artificial Sequence
  TrkAIg2 6His sequence 2
gccgcgcggcagc

SEQ ID NO: 40 - DNA, 13 bp, Artificial Sequence
  TrkAIg2 6His sequence 3
tatgccggccagt

SEQ ID NO: 41 - DNA, 11 bp, Artificial Sequence
  TrkAIg2 6His sequence 4
acggcggtggg

SEQ ID NO: 42 - DNA, 103 bp, Artificial Sequence
  TrkAIg2 noHis sequence
ccctctagaaataattttgtttaactttaagaaggagatatacatatgccggccagtgtgcagctgcacacggcggtggagatgcaccactggtgcatcccct

SEQ ID NO: 43 - DNA, 84 bp, Artificial Sequence
  TrkAIg2 noHis sequence 2
tctagaaataattttgtttaactttaagaaggagatatacatatgcctgcttcagtacaattacacacggcggtggagatgcac

SEQ ID NO: 44 - DNA, 18 bp, Artificial Sequence
  TrkAIg2 noHis sequence 3
tgcatccccttctctgtg

(19) United States
(12) Patent Application Publication
(10) Pub. No.: US 2005/0070690 A1
(43) Pub. Date: Mar. 31, 2005

(54) Polypeptide purification method

(76) Inventors: David Dawbarn, Bristol (GB); Shelly Jane Allen, Bristol Royal Infirmary Bristol (GB); Alan GeorgeSimpson Robertson, Cambridgeshire (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 10/489,739

(22) PCT Filed: Sep. 17, 2002
(86) PCT No.: PCT/GB02/04214

(30) Foreign Application Priority Data
Sep. 17, 2001 (GB) 0122400.5

(51) Int. Cl. C07K 14/705; C07H 21/04; C12N 9/12

(52) U.S. Cl. 530/350; 435/69.1; 435/320.1; 435/325; 435/194; 536/23.6

(57) ABSTRACT

This invention relates to a purification method, particularly for purifying tyrosine kinase receptor related polypeptides, and to products made by the method. The method comprises a method of producing tyrosine kinase receptor-related polypeptides, the method comprising expressing a tyrosine kinase receptor-related polypeptide in a recombinant expression system and separating expressed monomeric tyrosine kinase receptor-related polypeptide from multimeric form(s) of the expressed polypeptide in a separation step, the separation step allowing refolding of the expressed tyrosine kinase receptor-related polypeptide into a biologically active form.

### FIELD TO WHICH THE INVENTION RELATES

This invention relates to a method for purifying polypeptides and to products obtained by the method. In particular, though not exclusively, the invention relates to a method for purifying polypeptides which have to fold before they are biologically active such as the tyrosine kinase receptors TrkA, TrkB and TrkC and biologically active variants and portions thereof (all referred to as “tyrosine kinase receptor-related polypeptides”).

### BACKGROUND

The tyrosine kinase receptors TrkA, TrkB and TrkC bind neurotrophins. TrkA is biologically active in that it binds nerve growth factor (NGF) with high affinity. It is also biologically active in that it binds neurotrophin-3 (NT3) with high affinity. TrkB and TrkC bind other neurotrophins. TrkB binds brain derived neurotrophic factor (BDNF) and neurotrophin-4 (NT4) with high affinity. TrkC binds NT3 with high affinity. The identification, cloning and sequencing of TrkB and TrkC are described in U.S. Pat. No. 6,027,927. Each receptor molecule comprises a number of regions or domains. The immunoglobulin-like domains (Ig) of the tyrosine kinase receptor molecule are of particular interest in therapeutic applications. More particularly, as disclosed in the applicant's co-pending patent application, WO99/53055, TrkAIg₂ and variants, such as the splice variant TrkAIg_{2.6}, have therapeutic application.

There is a need to produce polypeptides derived from the tyrosine kinase receptors on a large scale, particularly for therapeutic applications. Production of recombinant polypeptides in bacterial expression systems is advantageous for several reasons, particularly because relatively high yields of polypeptide can be obtained. Typically yields can be ten times higher than in human cell systems.

However, the expressed polypeptides such as TrkAIg₂, the second immunoglobulin-like domain of TrkA, are difficult to work with in that they tend to be produced as a mixture of monomer, dimer and aggregate (i.e. aggregated dimer which may include monomer amongst the dimer) In particular in the case of TrkAIg₂, but also in the cases of TrkBIg₂ and TrkCIg₂, only the monomer is, however, active and therefore therapeutically useful. As discussed in Robertson A. G. S. et al (Biochemical and Biophysical Research Communications 282 (1): 131-141 Mar. 23, 2001) dimers of TrkAIg₂ are not able to bind NGF and are not biologically active. It is likely that small amounts of dimer or aggregate seed the production of more aggregate leading to a decrease in amount of biologically active monomer. This is unusual, many proteins exist in an equilibrium between monomer, dimer and even tetramer, (e.g. human growth hormone). The removal of dimer is crucial to a long term stable preparation, which is a requirement for pharmaceutical formulation. Like many proteins, correct conformation of the tyrosine receptor-related polypeptide is important for biological activity. When expressed in a bacterial inclusion body the polypeptide is folded, but in an incorrect biologically inactive conformation. After expression in a recombinant system the polypeptide must be folded again to achieve that correct conformation. This further folding after expression is sometimes referred to as “refolding”.

We are only aware of two other groups that have tried to make recombinant TrkAIg₂ in bacterial cells. Ultsch et al (J Mol Biol (1999) 290, 149-159) made TrkAIg₂, TrkBIg₂ and TrkCIg₂. They made the TrkAIg₂ as soluble protein, rather than in inclusion bodies, purified it by ion exchange, then hydrophobic interaction, ion exchange again and gel filtration. The gel filtration step here was not to allow refolding of the polypeptide—it would be assumed that the polypeptide was already correctly folded as it was in soluble form. TrkBIg₂ and TrkCIg₂, although expressed in the same way, were insoluble. They were solubilised in urea and dialysed to allow refolding and further purified by ion exchange. Solution of crystal structures of the resulting TrkAIg₂, TrkBIg₂ and TrkCIg₂ revealed strand swapped dimers i.e. dimers where strand A of one monomer is paired with strand B of another monomer (see the accompanying FIG. 1). FIG. 1 shows a strand swapped dimer consisting of two TrkAIg₂ monomers in which the A strand from monomer X unfolds and binds with the B strand from monomer Y. Conversely the A strand from monomer Y unfolds and binds with the B strand from monomer X. These are inactive. In their discussion it was indicated that none of the dimers produced were capable of binding to the natural ligands, in contrast to the domains expressed as immunoadhesins in 293 cells (Urfer et al (1995) EMBO Journal 14, 12, p2795-2805). The apparent NGF binding activity of the TrkAIg₂—immunoadhesin molecule constructs in animal cells was probably due to the large immunoadhesion scaffold (the F_{c} portion of IgG) holding the TrkAIg₂ region in a correct conformation, which would lead to extensive glycosylation, and would also be likely to significantly affect the binding of the construct to other molecules.

Windisch et al (Windisch, J M. et al (1995) J. Biol. Chem. 270 47 p28133-28138) produced TrkA derivatives including TrkAIg₂ as maltose binding protein fusion constructs which were inactive and therefore would not be suitable for therapeutic use. Maltose binding protein constructs are used with “difficult” proteins. It was assumed that the constructs had folded correctly but it is now apparent that this was not the case.

Wiesmann et al (Nature, 9 Sep. 1999 401, 184-188) could only produce a co-crystal of NGF and TrkAIg₂ by adding together NGF and TrkAIg_{1,2} i.e. a polypeptide comprising both Ig-like domains of TrkA. Over a period of many months the TrkAIg₁ region was ‘nibbled’ away leaving only the TrkAIg₂ region bound to the NGF. Such a method is not suitable for commercial level production of tyrosine kinase receptor related polypeptides.

One method of producing biologically active portions and derivatives of tyrosine kinase receptor-related polypeptides in inclusion bodies in Escherichia coli is disclosed in Holden et al (Holden, P. H. et al Nature Biotechnology, 15, July 1997 page 668-672). This method involves a dialysis step, after extraction of the polypeptide from the inclusion bodies, to allow the expressed polypeptide to refold, and results in a yield of only about 16 mg/litre of polypeptide. WO99/53055 discloses a similar method of purifying portions and derivatives of TrkA, including TrkAIg₂, from E. coli inclusion bodies in which the extracted polypeptide is also dialysed. This method leads to a yield of about ˜50 mg/litre. This method produces a product which is relatively unstable, having to be snap frozen after production, and before it can be used further.

As noted above, the methods described in Ultsch et al, WO99/53055 and in Holden et al involve a dialysis step. Dialysis is relatively disadvantageous in that it requires large amounts of dialysis buffer in order to limit the concentration of polypeptide (usually to about 0.1 mg/ml) and to limit aggregation of the polypeptide. The amount of dialysis buffer involved precludes the use of a method of producing tyrosine kinase receptor-related polypeptides involving dialysis on a commercially-useful scale.

It is an object of the present invention to provide a method of producing polypeptides, particularly tyrosine kinase receptor-related polypeptides, which provides improved yields compared to prior art processes. It is a further object of the present invention to provide a method which provides a product with improved stability compared to prior art processes.

### SUMMARY OF THE INVENTION

According to one aspect of the invention there is provided a method of producing tyrosine kinase receptor-related polypeptides, the method comprising expressing a tyrosine kinase receptor-related polypeptide in a recombinant expression system and separating expressed monomeric tyrosine kinase receptor-related polypeptide from multimeric form(s) of the expressed polypeptide in a separation step, the separation step allowing refolding of the expressed tyrosine kinase receptor-related polypeptide into a biologically active form.

The method is advantageous over known processes for several reasons. First, the method produces significantly higher yields than known processes. Second, the method is scalable allowing production of polypeptide at commercially useful levels. Third, the method does not require a separate dialysis-based refolding step. Dialysis requires large amounts of expensive dialysis buffer since it requires refolding at low polypeptide concentrations, and a lengthy period of time for refolding. Methods involving a dialysis step require a recovery step for capture of the polypeptide. This can be done for instance by ion exchange or affinity separation. The use of ion exchange requires increased levels of NaCl to elute product; this is disadvantageous in that it causes further aggregation of the polypeptide. The use of affinity separation for example using a His tag on a nickel chelating column also requires relatively high NaCl levels and elution with imidazole requires gel filtration to remove. Fourth, the process is much quicker than prior art processes involving dialysis, which is usually done overnight. Fifth, the product of the method is more stable. Rather than having to be snap frozen immediately after production, it can be kept normally refrigerated (at about 4° C.) and is biologically active for at least three months. As the product has lower dimer levels there is less tendency for aggregation seeded by dimers to take place. Sixth, the product can be produced at higher concentrations (up to 650 μM) without strand swap dimers being produced. Seventh, as the polypeptide product is not in contact with urea for the lengthy periods required during a dialysis procedure it is less likely to be amidated. Amidation can affect biological activity. It may also make it more difficult to couple the polypeptide to matrices using amine coupling methods. This makes products of the method of the present invention more useful in certain applications such as biosensors.

The tyrosine kinase receptor may be native TrkA, TrkB, TrkC; or a biologically active homologue, variant, portion of those receptors or a construct including a homologue, variant, or portion thereof. Preferably the polypeptide is selected from TrkAIg₂ and TrkBIg₂. Particularly preferred polypeptides for production by the method of the present invention are the Ig₂ subdomains of the TrkA, TrkB, and TrkC receptors. Most preferably the polypeptide is TrkAIg₂ or TrkAIg_{2.6}.

Preferred constructs may include additional C terminal sequence from the corresponding native receptor.

The polypeptide may be expressed with a histidine tag sequence. The tyrosine kinase sequence is preferably human. The tyrosine kinase receptor-related polypeptide may be expressed in insoluble form. Preferably the tyrosine kinase receptor-related polypeptide is expressed in bacterial inclusion bodies. The multimeric forms of the polypeptide may include dimers. The polypeptide is preferably able to bind a ligand of the corresponding native tyrosine kinase receptor with high affinity.

The separation step preferably involves gel filtration. The separation step is preferably carried out at a salt concentration between 0 mM and 500 mM, and more preferably above 25 mM and below 200 mM, most preferably at a salt concentration of about 100 mM, for example in the range 80 mM to 120 mM. The gel used in the gel filtration step is preferably able to separate molecules having a molecular weight of about 12 to 40 kDa The gel may be for example Sephacryl 200, SuperDex 75 or SuperDex 200.

The separation step is preferably carried out at an alkaline pH. Preferably, the separation step is carried out at a pH below one where denaturation occurs. For example, the step may be carried out at typically between pH 8 and 9. Most preferably, the filtration step is carried out at about pH 8.5. This is unexpected in the case of TrkA as TrkAIg₂ has a calculated Pᵢ of between 4.6 and 6.0 dependant on the program used for the calculation.

TrkAIg₂ has a high level of β sheet and most proteins like this aggregate and precipitate near their pI. TrkAIg₂, however, precipitates and aggregates at pH's around physiological pH, and significantly different from its pI and at salt concentrations that normally maintain such proteins in solution.

In a preferred arrangement, polypeptide is eluted from the gel filtration step at a flow rate of about 2.5 ml/min, and monomer is collected after about 93 minutes. This will, however, vary according to the apparatus and conditions under which it is operated. Preferably, the polypeptide is produced in a bacteria-based expression system.

According to a preferred aspect of the invention there is provided a method of purifying recombinant TrkAIg₂ or TrkAIg_{2.6} from inclusion bodies in a bacterial expression system in which monomeric TrkAIg₂ is separated from a mixture including monomeric and multimeric TrkAIg₂ by a gel filtration step and allowed to refold into a biologically active form. Typically, the multimeric TrkAIg₂ will comprise dimeric TrkAIg₂.

The invention also provides a stable preparation of TrkAIg₂ obtained, or obtainable, by a method according to the invention and comprising less than 20% of TrkAIg₂ dimer or dimer aggregate, more preferably less than 1% of TrkAIg₂ dimer or dimer aggregate, most preferably less than 0.1% of TrkAIg₂ dimer or dimer aggregate.

The invention also provides a stable preparation of TrkAIg₂ obtained, or obtainable, by a method according to the invention and comprising more than 80% TrkAIg₂ monomer, more preferably more than 99% TrkAIg₂ monomer, most preferably 100% TrkAIg₂ monomer. Preferably, the monomer is substantially all in a biologically active form.

The invention also provides a preparation of TrkAIg_{2.6} obtained, or obtainable, by a method according to the invention and comprising less than 20% of TrkAIg_{2.6} dimer or dimer aggregate, more preferably less than 1% of TrkAIg_{2.6} dimer or dimer aggregate, most preferably less than 0.1% of TrkAIg₂ dimer or dimer aggregate.

The invention also provides a stable preparation of TrkAIg_{2.6} obtained or obtainable, by a method according to the invention and comprising more than 80% TrkAIg_{2.6} monomer, more preferably more than 99% TrkAIg_{2.6} monomer, most preferably 100% TrkAIg_{2.6} monomer. Preferably, the monomer is substantially all in a biologically active form.

The invention also provides a stable preparation of TrkBIg₂ obtained, or obtainable, by a method according to the invention comprising less than 20% of TrkBIg₂ dimer or dimer aggregate, more preferably less than 1% of TrkBIg₂ dimer or dimer aggregate, most preferably less than 0.1% of TrkBIg₂ dimer or dimer aggregate.

The invention also provides a stable preparation of TrkBIg₂ obtained, or obtainable, by a method according to the invention comprising more than 80% TrkBIg₂ monomer, more preferably more than 99% TrkBIg₂ monomer, most preferably 100% TrkBIg₂ monomer. Preferably, the monomer is substantially all in a biologically active form.

The invention also provides a stable preparation of TrkCIg₂ obtained, or obtainable, by a method according to the invention and comprising less than 20% of TrkCIg₂ dimer or dimer aggregate, more preferably less than 1% of TrkCIg₂ dimer or dimer aggregate, most preferably less than 0.1% of TrkCIg₂ dimer or dimer aggregate.

The invention also provides a stable preparation of TrkCIg₂ obtained, or obtainable, by a method according to the invention and comprising more than 80% TrkCIg₂ monomer, more preferably more than 99% TrkCIg₂ monomer, most preferably 100% TrkCIg₂ monomer. Preferably, the monomer is substantially all in a biologically active form.

According to another aspect of the invention there is provided a method of producing immunoglobulin-like polypeptide monomers from a mixture of monomeric and multimeric forms of the polypeptide, the method comprising expressing the polypeptide in a recombinant expression system and separating polypeptide monomers from multimeric forms of the polypeptide in a separation step, the separation step allowing the polypeptide to refold to a biologically active form. Thus the invention provides a method of purifying immunoglobulin-like polypeptides which has some or all of the advantages described above. The separation step preferably includes gel filtration.

### BRIEF DESCRIPTION OF THE DRAWINGS

Methods and products in accordance with the invention will now be described, by way of example only, with reference to the further accompanying FIGS. 2 to 22 in which:

FIG. 2 shows the amino acid sequences of (A) TrkAIg₂ and TrkAIg_{2.6}; (B) TrkBIg₂ truncated and full length forms (in bold; pET15b sequences (MGSSHHHHHH SSGLVPRGSHM) in unbolded form); and (C) TrkCIg₂ truncated and full length forms (in bold; pET15b sequences (MGSSHHHHHH SSGLVPRGSHM) in unbolded form);

FIG. 3 is a overlap of traces from an FPLC machine illustrating comparative experiments with a prior art dialysis method and a method in accordance with the invention;

FIG. 4 is a series of traces illustrating the results of experiments in which pH was altered;

FIG. 5 is a series of traces illustrating comparative experiments with volume of dialysis buffer;

FIG. 6 shows results of mass spectrometry experiments on TrkAIg₂6His and TrkAIg_{2.6}6His produced by the invention;

FIG. 7 illustrates the results of binding activity studies for TrkBIg₂6His, with A: BDNF and B: NT4;

FIG. 8 illustrates the results of binding activity studies with TrkAIg₂6His with NGF;

FIG. 9 illustrates the results of binding activity studies with TrkAIg_{2.6}6His with NGF;

FIG. 10 shows results of mass spectrometry experiments on TrkBIg₂6His produced by the invention;

FIG. 11 shows results of PC12 cell neurite outgrowth bioassay using TrkAIg₂6His;

FIG. 12 shows result of mass spectrometry experiments on TrkCIg₂6His produced by the invention;

FIG. 13 illustrates the results of binding activity studies with TrkCIg₂6His with NT-3;

FIG. 14 illustrates the predicted mRNA structure of TrkAIg₂6His;

FIG. 15 illustrates the predicted mRNA structure of TrkAIg₂ noHis;

FIG. 16 illustrates an example of mutations required to facilitate expression of TrkAIg₂noHis;

FIG. 17 illustrates the predicted mRNA structure for the mutant sequence shown in FIG. 16;

FIG. 18 shows an SDS-PAGE gel showing cell extracts from E. coli expressing the pET24a-TrkAIg₂ noHis mutant sequence shown in FIG. 16;

FIG. 19 shows results of mass spectrometry experiments on TrkAIg₂ noHis produced by the invention;

FIG. 20 shows results of PC12 cell neurite outgrowth bioassay using TrkAIg₂ noHis;

FIG. 21 illustrates examples of mutations required to facilitate expression of TrkBIg₂noHis; and

FIG. 22 illustrates examples of mutations required to facilitate expression of TrkCIg₂noHis.

Definitions:

“Polypeptide”: This term embraces proteins i.e. naturally occurring full length biologically active polypeptides.

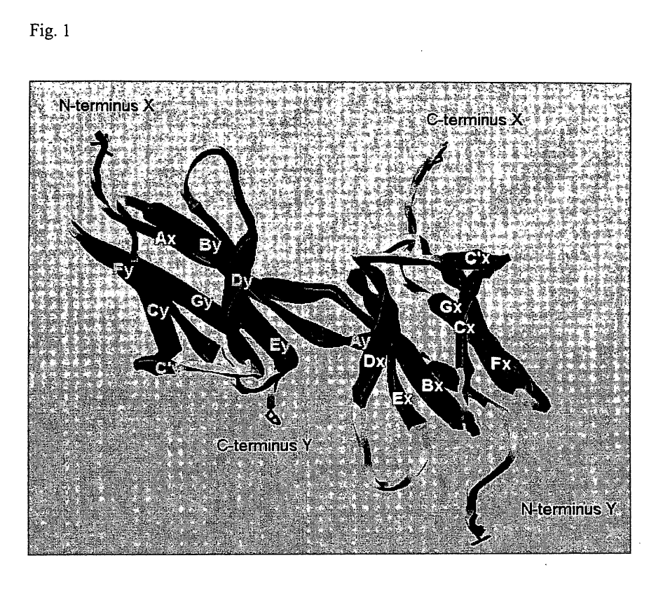

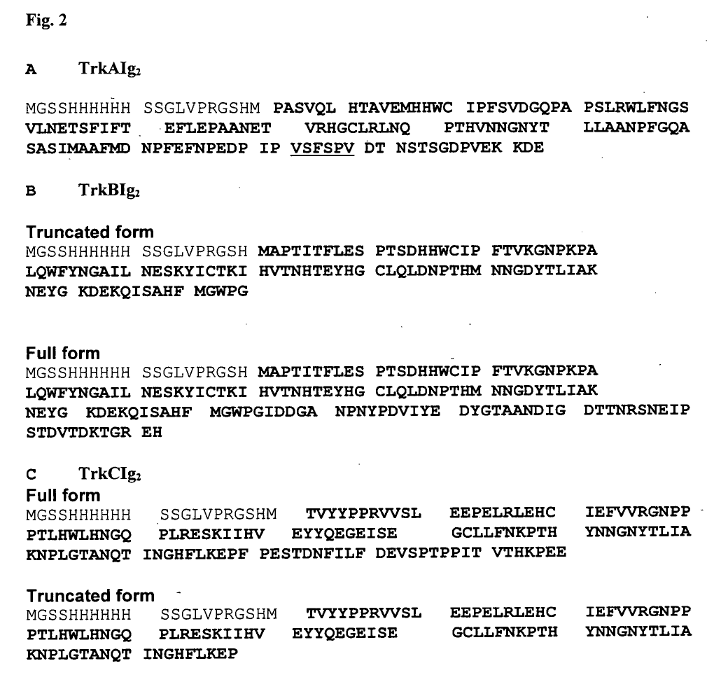

“TrkAIg₂”: is a polypeptide having the amino acid sequence shown in bold in FIG. 2A (whether with or without the additional six amino acid residues underlined which lead to the variant TrkAIg_{2.6}) and homologues (for example as a result of conservative substitutions of one or more amino acid residues in the sequence) or variants including sequences to enhance expression and/or purification such as the his tag and thrombin cleavage sequence shown in unbolded form in FIG. 2A.

“TrkBIg₂” and “TrkCIg₂” have corresponding meanings with reference to the sequences shown in FIGS. 2B and 2C respectively.

“TrkAIg₂6His” represents variants including the His tag and “TrkAIg₂ noHis” represents variants not including the His tag. Similar terms apply to corresponding variants of TrkB and TrkC and proteins thereof.

Specific Description

Production of Histidine-Tagged TrkIgs

Production of Recombinant TrkAIg₂6His and TrkAIg_{2.6}6His Polypeptide

Recombinant TrkAIg₂6His was produced in E. coli BL21 (DE3) cells using the method described in WO99/53055 in the section headed “Expression of TrkAIg_{1,2}, TrkAIg₁ and TrkAIg₂” and incorporating a 6-histidine tag to the N-terminus of the polypeptide as shown in FIG. 2A. Recombinant TrkAIg_{2.6} 6His was prepared in a similar manner.

Purification and Refolding of TrkAIg₂6His Polypeptide

The harvested cells were resuspended in 10% glycerol, frozen at −70° C. in liquid nitrogen and the resulting pellet was passed three times through an XPress (AB Biox). The extract was centrifuged at 10,000 rpm, 4° C. for 30 min to pellet the insoluble inclusion bodies containing the recombinant polypeptide.

The inclusion bodies were washed in 500 ml 1% (v/v) Triton X-100, 10 mM Tris HCl pH8.0, 1 mM EDTA followed by 500 ml 1M NaCl, 10 mM Tris HCl pH 8.0, 1 mM EDTA and finally 10 mM Tris HCl pH8.0, 1 mM EDTA.

The inclusion bodies were then solubilised in 20 mM Na Phosphate, 30 mM Imidazole, 8M Urea (pH 7.4) and clarified by centrifugation. 6M Guanidinium may also be used in place of 8M urea throughout.

The resulting mixture was loaded on a 5 ml HisTrap column(Pharmacia), and washed with 50 ml 20 mM NaPhosphate, 30 mM Imidazole, 8M Urea pH 7.4. The purified TrkAIg₂6His was eluted with 25 ml 20 mM NaPhosphate, 300 mM Imidazole, 8M Urea (pH7.4).

In order to allow the purified recombinant TrkAIg₂6His polypeptide to refold, the eluant of the previous step was then applied to a SuperDex 200 gel filtration column, and equilibrated in 20 mM NaPhosphate, 100 mM NaCl, at pH 8.5. The column had a height of 65 cm, width 2.6 cm and volume of 345 ml when pre-packed by the manufacturer. The flow rate at maximum pressure was 2.5 ml/min.

Any aggregate (i.e. dimer aggregates) eluted in the void volume. Dimer was eluted after about 80 min and monomer eluted after about 93 min. The elution time of a protein will be dependent on dimensions of column and is dependent on its size. This is described by the following formula:
R=VO/Ve
where R is retention coefficient of a protein, Ve is the volume at which the protein is eluted, VO is void volume
where Ve=232.5 ml VO is 122 ml
122/232.5=0.53

TrkAIg₂6His monomer on a SuperDex 200 gel has a retention coefficient of 0.53.

By way of comparison the polypeptide was also folded by dialysis first against 20 mM Tris HCl, 50 mM NaCl, pH 8.5, recaptured on a His Trap column and eluted with 25 ml 20 mM NaPhosphate, 300 mM Imidazole, 8M Urea pH 7.4.

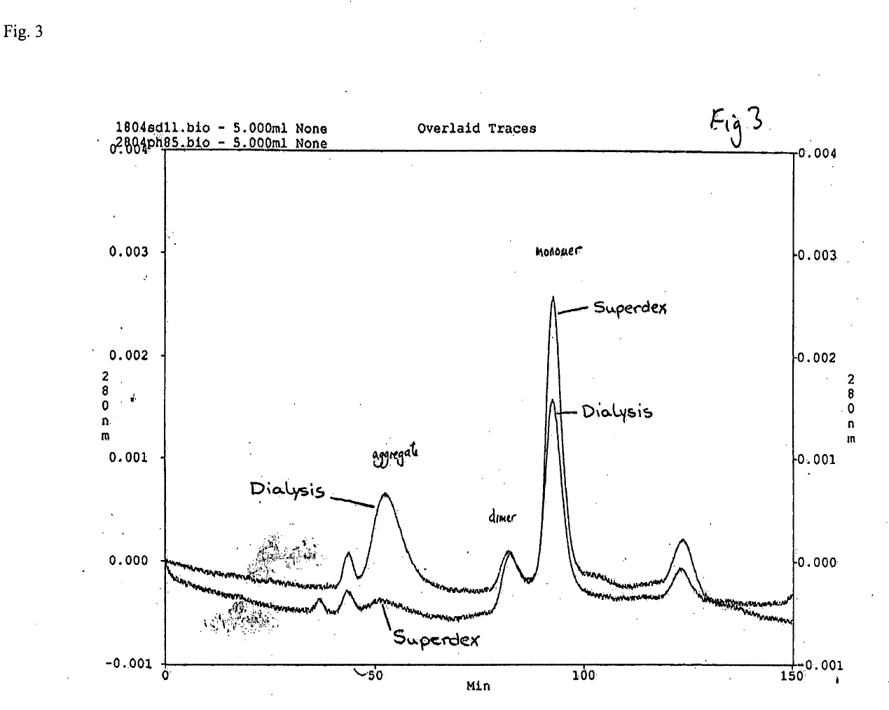

Purification traces from the Biocad Sprint FPLC (Biocad) which show elution of monomer, dimer and aggregates of TrkAIg₂6His under various conditions were prepared. FIG. 3 shows an overlay trace comparing elution of TrkAIg₂6His with refolding by dialysis (“Dialysis”) and with refolding on a column in a method according to the invention (“SuperDex”). It will be seen that the method of the invention produces higher levels of monomer compared to the prior art process.

The splice variant TrkAIg_{2.6}6His was prepared and purified in a similar manner.

Effect of pH on Elution of TrkAIg₂6His

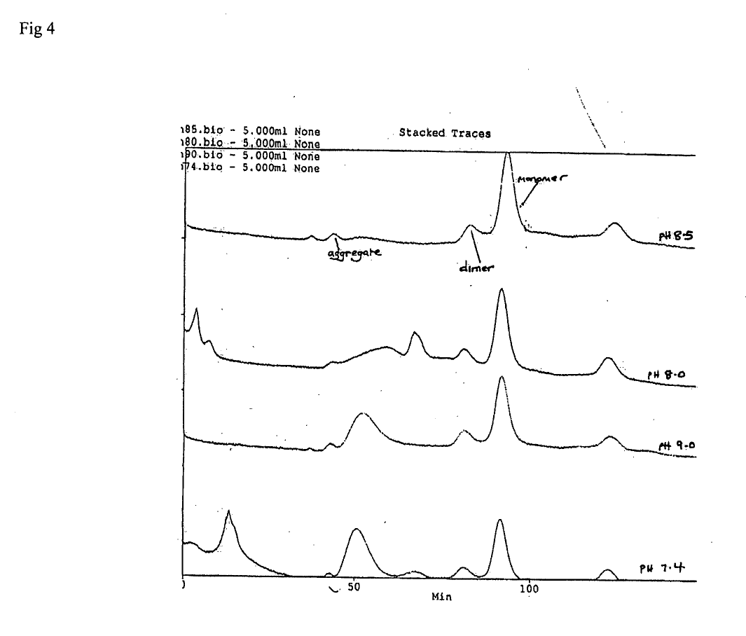

TrkAIg₂6His was expressed in E. coli as described above. Purified inclusion bodies were solubilised in 20 mM Na Phosphate, 30 mM Imidazole, 8M Urea pH 7.4 and clarified by centrifugation. The resulting mutant was affinity purified on HisTrap column and eluted with 25 ml 20 mM NaPhosphate, 300 mM Imidazole, 8M Urea pH 7.4. The eluted TrkAIg₂6His was applied to SuperDex 200 gel filtration column (Pharmacia) and equilibrated in 20 mM NaPhosphate, 100 mM NaCl, pH 8.5. The flow rate was 2.5 ml/min. The time taken for elution is determined by size of protein. Peaks of monomer, dimer and aggregate are indicated at approximately 93 minutes, 80 minutes and 50 minutes respectively. The results are given in FIG. 4 whichshows the results of elution at pH7.4, 8.0, 8.5 and 9.0. The results indicate that pH 8.5 was best with the greatest yield, lowest amount of aggregate, and highest levels of monomer.

Comparative Example: Separation of TrkAIg₂6His Monomer, Dimer and Aggregate with Refold by Dialysis. Amount of Dialysis Buffer Required. Subsequent Analysis by Elution from SuperDex 200.

TrkAIg₂6His was expressed in E. coli. Purified inclusion bodies were solubilised in 20 mM NaPhosphate, 30 mM Imidazole, 8M Urea pH 7.4 and clarified by centrifugation. The solution was affinity purified on HisTrap column and eluted with 25 ml 20 mM NaPhosphate, 300 mM Imidazole, 8M Urea pH 7.4. TrkAIg₂6His was folded by dialysis (using 1 litre, 2 litres or 4 litres) overnight against 20 mM Tris HCl, 50 mM NaCl, pH 8.5, recaptured on a HisTrap column and eluted with 25 ml 20 mM NaPhosphate, 50 mM EDTA, 8M Urea pH 7.4. Final analysis was using a SuperDex 200 gel filtration column equilibrated in 20 mM NaPhosphate, 100 mM NaCl, pH 8.5. Flow rate was 2.5 ml/min. 2-4 litres were required for washing. 4 litres gave the highest yield of monomer. This shows how large volumes of buffer are needed if dialysis is to be used for refolding of the expressed polypeptide.

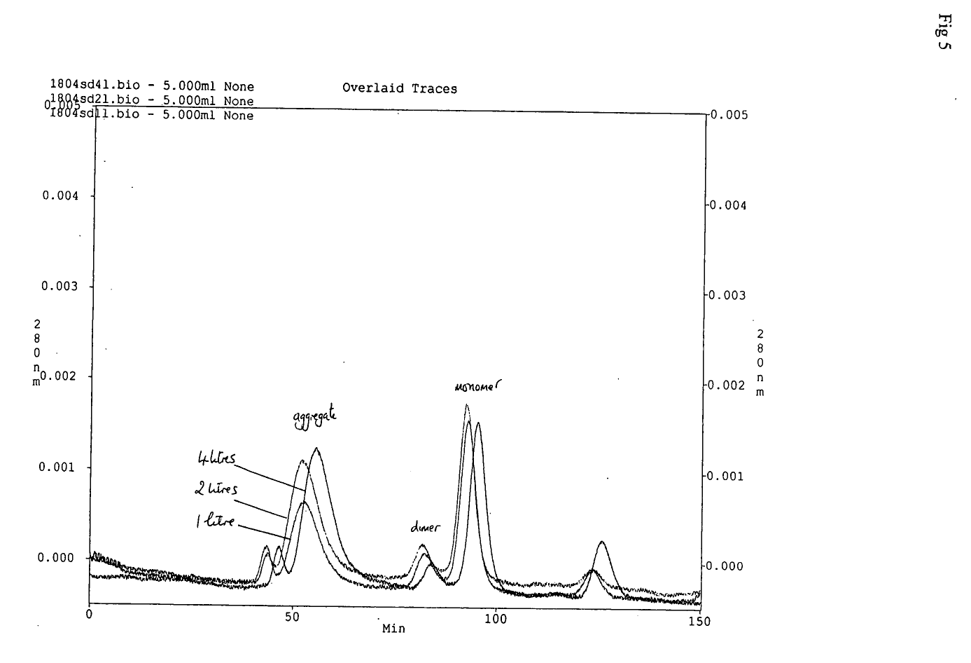

Results are shown in FIG. 5.

Characterisation of TrkAIg₂6His and TrkAIg_{2.6}6His Produced by Method of the Invention

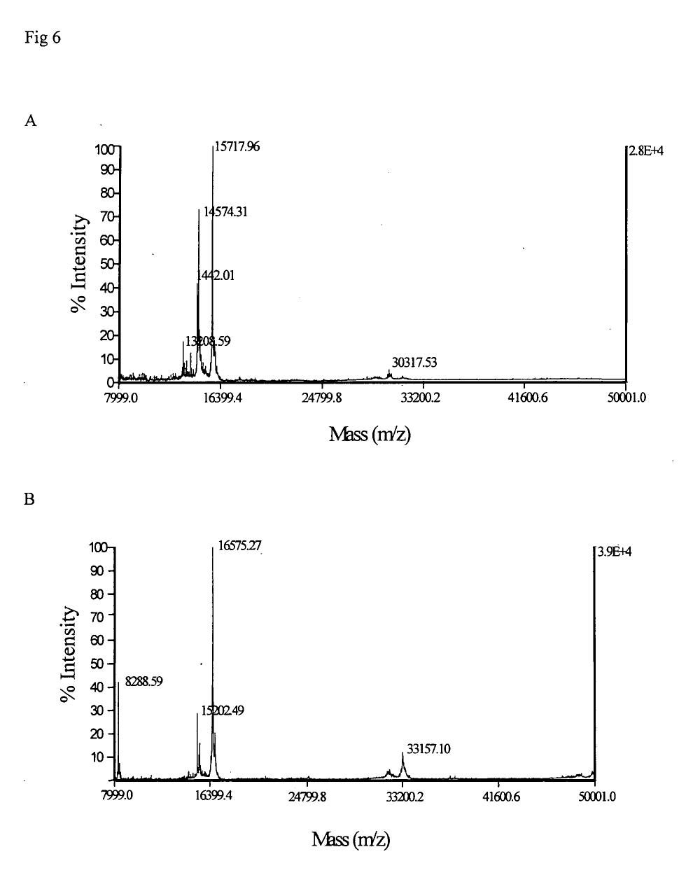

The expressed TrkAIg₂6His (A) and TrkAIg_{2.6}6His (B) polypeptides were subjected to MALDITOF mass spectrometry and the results are shown in FIG. 6. The molecular mass of the polypeptides was determined using a PE Biosystems Voyager-DE STR Matrix-Assisted Laser Desorption Time-of-Flight (MALDITOF) mass spectrometer with a nitrogen laser operating at 337 nm. The matrix solution was freshly prepared sinapinic acid at a concentration of 1 mg/1001 μl in a 50:50 mixture of acetonitrile and 0.1% trifluoroacetic acid. 0.51 μl of sample and matrix were spotted onto the sample plate. The sample was calibrated against Calmix 3 (PE Biosystems) run as a close external standard. The spectrum was acquired over the range 5000-80,000 Da, under linear conditions with an accelerating voltage of 25,000 V, an extraction time of 750 nsecs and laser intensity of 2700.

The molecular weight of TrkAIg₂6His was found to be 15,717.96 Da. This is almost exactly as predicted by theoretical calculation of the molecular weight (15716.3 Da, after loss of the N-terminal methionine, which we have previously found to be removed in proteins incorporating the 6 histidine tag from the expression vector pET15b).

The molecular weight of TrkAIg_{2.6}6His was found to be 16,575.3 Da. This is almost exactly as predicted by theoretical calculation of the molecular weight (16,574.4 Da).

Stability

TrkAIg₂6His and TrkAIg_{2.6}6His produced as described above has remained stable when kept at 4° C. for three months and has retained its biological activity.

Improvements in stability may be achieved using conventional additives such as glycerol.

Biological Activity of TrkAIg₂6His Produced by the Method of the Invention

i Guinea Pig Hind Limb Pain Responses

Biological activity of TrkAIg₂6His produced by the method of the invention was tested in guinea pigs (Djouhri, L. et al (2001) J Neuroscience 21 p8722-8733). CFA (Complete Freund's Adjuvant) was injected into the hind limb and knee of guinea pigs. This causes inflammation which leads to an increase of NGF levels. This makes the animal more susceptible to feeling pain. Intracellular recordings were made from the cell bodies of L6 (lumbar), and S1 (sacral) DRG neurons with glass microelectrodes and action potentials were evoked by stimulation of DRG with a pair of platinum electrodes. The recordings were made 1, 2 and 4 days after CFA administration. The C and Aδ fibres are nociceptive —they transmit pain signals to the brain. α and β fibres do not. Spontaneous firing of nociceptive neurons without outside stimulation is thought to be responsible for inflammatory and neuropathic pain in humans.

TrkAIg₂6His was injected on days 2, 3 and 4 with 0.45 μg into hind limb and knee on guinea pig. Adding TrkAIg₂6His, which sequesters the endogenous NGF, abolished CFA-induced increases in following frequency and in spontaneous firing. This meant complete cessation of abnormal pain.

TrkAIg₂6His was therefore able to inhibit pain response in CFA induced pain fibre firing in guinea pigs.

ii PC12 Cell Bioassays

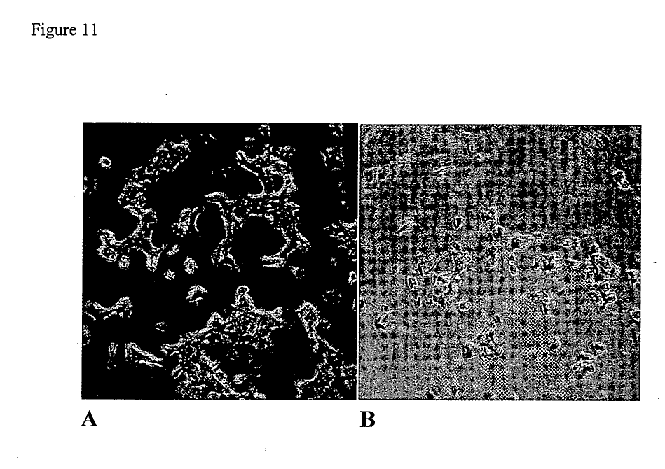

Biological activity of TrkAIg₂6His produced by the method of the invention was tested by PC12 neurite outgrowth bioassay. PC12 cells are a rat phaeochromocytoma cell line which grow neurites in response to the presence of NGF, which binds receptors present on the cell surface. PC12 cells were plated out at 2×10⁴ cells per well in complete DMEM medium (including 100 units/ml penicillin, 100 μg/ml streptomycin, 10% horse serum, 10% Foetal Calf Serum (FCS) and 2 mM glutamine) on collagen-coated 24-well plates. NGF was added at 1 ng/ml and TrkAIg₂6His was added at varying concentrations. Results are shown in FIG. 11, photographs of neurite outgrowth after 48 hours. Cells were fixed before photographing. FIG. 11A shows neurite outgrowth with 1 ng/ml NGF and FIG. 11B shows no neurite outgrowth when 1.25 μm TrkAIg₂6His is added.

TrkAIg₂6His was therefore able to prevent neurite growth in response to NGF in the PC12 cell line.

Sub-Cloning of the TrkBIg₂6His Domain

The TrkBIg₂6His protein comprises residues 286 to 430 of the mature protein, and has a further 21 residues at the NH₂ terminus which constitute the histidine expression tag and associated thrombin cleavage sequence. cDNA coding for the TrkBIg₂₆His domain was PCR amplified from λZAP-pBluescriptllSK⁽⁻⁾/TrkB, a non-catalytic form of human TrkB cloned by us (Allen et al (1994) Neuroscience 60 p825-834). Primers (MWG Biotech) incorporated a Nde1 site in the forward primer (CGCATATGGCACCAACTATCACATTTCTCGAATCTC), and a BamHI site in the reverse primer:

(GCGGATCCCTATTAATGRRCCCGACCGGTTTTATC).

The PCR product was subcloned into pET15b (Novagen), using Nde1 and BamHI sites, to create the expression vector pET15b-TrkBIg₂6His.

A truncated version of TrkBIg₂6His, shown in FIG. 2B, was also produced in exactly the same way but using the amino acids 286-383. This form was co-crystallised with its ligand NT4 and an X-ray crystal structure solved.

Production of Recombinant TrkBIg₂6His Polypeptide

Electro-competent E. coli BL21 (DE3) cells were transformed with pET15b-TrkBIg₂, and expression was carried out in accordance with the pET (Novagen) manual. After transformation, E. coli cell lysates were analysed by SDS-PAGE for expression of the 18.5 kDa protein. TrkBIg₂6His protein was expressed at high levels in the urea-soluble fraction, but not in the other fractions. 2 ml of 2YT broth (containing 200 μg/1 ml ampicillin) was inoculated with a colony and grown at 37° C. to mid log phase. This was used to inoculate 50 ml of 2YT broth (containing 200 μg/ml ampicillin), which was grown at 37° C. to mid log phase. This was used to inoculate 5 litres of 2YT broth (containing 200 μg/ml ampicillin), which was grown to an optical density of 1.0 at 600 nm. 1 mM IPTG was added to induce protein expression and cells were grown overnight at 37° C. The harvested cells were resuspended in 10% glycerol and frozen at −80° C. (8 pellets). Pellets were lysed by passing 3 times through an Xpress, then washed with 20 mM sodium phosphate buffers (pH 8.5) containing, in succession, 0.1M NaCl, 1% Triton X-100, and finally 1M NaCl. This removed all soluble matter, leaving inclusion bodies containing insoluble protein.

Refolding of TrkBIg₂6His Polypeptide

Insoluble TrkBIg₂6His protein contained in the inclusion bodies was released from the cells with an Xpress, and washed to remove soluble matter. The purified inclusion bodies were solubilised in 8M urea buffer (20 mM sodium phosphate, pH 8.5, 1 mM β-mercaptoethanol), with a “Complete” proteinase inhibitor cocktail tablet (Roche) and incubated at room temperature for 2 hours with gentle shaking. 6M Guanidinium may be substituted for 8M urea. TrkBIg₂6His protein was purified on a HisTrap nickel column (Pharmacia), under reducing conditions (20 mM sodium phosphate, pH 8.5, 8M urea, 10 mM imidazole), and eluted using 300 mM imidazole. Refolding took place under non-reducing conditions (20 mM sodium phosphate, pH 8.5, 100 mM NaCl) on a SuperDex 200 gel-filtration column (Pharmacia). Fractions from the peak corresponding to a molecular weight of approximately 18.5 kDa were pooled; these contained TrkBIg₂6His monomer.

Characterisation of TrkBIg₂6His Produced by Method of the Invention

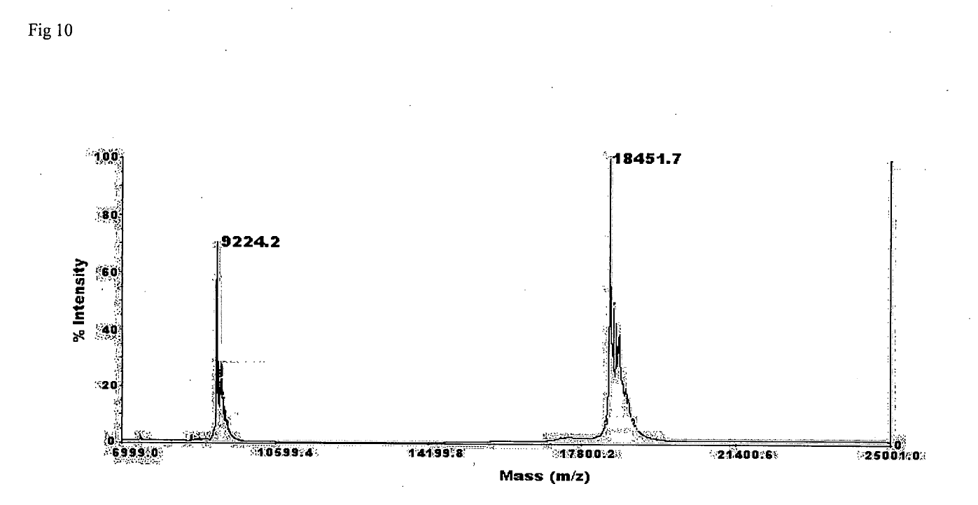

The molecular mass of TrkBIg₂6His was determined using a PE Biosystems Voyager-DE STR MALDITOF mass spectrometer, with a nitrogen laser operating at 337 nm. The matrix solution was freshly prepared sinapinic acid at a concentration of 1 mg/100 μl in a 50:50 mixture of acetonitrile and 0.1% trifluoroacetic acid. 0.5 μl of sample and matrix were spotted onto the sample plate. The sample was calibrated against Calmix 3 (PE Biosystems) run as a close external standard. The spectrum was acquired over the range 5000-80,000 Da, under linear conditions with an accelerating voltage of 25,000 V, an extraction time of 750 ns and a laser intensity of 2700. Results are shown in FIG. 10.

The molecular weight of TrkBIg₂₆His was found to be 18,451.7 Da This is almost exactly as predicted by theoretical calculation of the molecular weight (18,449.1 Da).

Sub-Cloning of Recombinant TrkCIg₆His Domain

The TrkCIg₂6His protein comprises residues 300 to 399 of the mature protein, and has a further 21 residues at the NH₂ terminus which constitute the histidine expression tag and associated thrombin cleavage sequence. cDNA coding for the TrkCIg₂6His domain was PCR amplified using a forward primer which incorporated a Nde1 site (CGCATATGACTGTCTACTATCCCCCAC) and a reverse primer which incorporated a BamH1 site (GCGGATCCTTATCAGGGCTCCTTGAGGAAGTGGC). The PCR product was subcloned into pET15b (Novagen) using Nde1 and BamH1 restriction sites, to create the expression vector pET15b-TrkCIg₂6His.

Production of Recombinant TrkCIg₂₆His Polypeptide

Electrocompetent E. coli BL21 (DE3) cells were transformed with pET15b-TrkCIg₂6His and expression was carried out in accordance with pET (Novagen) manual. After transformation E. coli lysates were anaylsed by SDS-PAGE for expression of the 13.8 kDa protein. TrkCIg₂6His protein was expressed at high levels in the urea-soluble fraction but not in other fractions. 2 ml of 2YT broth (containing 200 μg/ml ampicillin), was inoculated with a colony which was grown at 37° C. to mid log phase. This was used to inoculate 50 ml of 2YT broth (containing 200 μg/ml amplicillin) which was grown at 37° C. to mid log phase. This was used to inoculate 5 litres of 2YT broth (containing 200 μg/ml ampicillin), which was grown to an optical density of 1.0 at 600 nm. 1 mM IPTG was added to induce protein expression and cells were grown overnight at 37° C. The harvested cells were resuspended in 10% glycerol and frozen at −80° C. (8 pellets). Pellets were lysed by passing 3 times through an Xpress, and then washed with 20 mM sodium phosphate buffer (pH8.0) containing, in succession, 0.1M NaCl, 1% Triton X-100, and finally 1M NaCl. This removed all soluble matter, leaving inclusion bodies containing insoluble protein. All washes were at 4° C.

Refolding of TrkCIg₂6His Polypeptide

Insoluble TrkCIg₂6His protein contained in the inclusion bodies was released from the cells with Xpress, and washed to remove soluble matter. The purified inclusion bodies were solubilised in 8M urea buffer (20 mM sodium phosphate pH 8.0, 1 mM β-mercaptoethanol) and incubated at room temperature for 2 hours with gentle shaking. 6M Guanidinium may be substituted for 8M urea. TrkCIg₂₆His protein was purified on a HisTrap nickel column (Pharmacia) in 20 mM sodium phosphate, pH 8.0, 8M urea, 10 mM imidazole, 1 mM β-mercaptoethanol and eluted using 300 mM imidazole. Refolding was in 20 mM sodium phosphate, pH 8.0, 100 mM NaCl, 1 mM β-mercaptoethanol on a SuperDex 200 gel filtration column (Pharmacia). Fractions from the peak corresponding to a molecular weight of approximately 13.8 kDa were pooled. These contained TrkCIg₂6His monomer. The retention coefficient of TrkCIg₂6His is 0.51.

Characterisation of TrkCIg₆His Produced by Method of the Invention

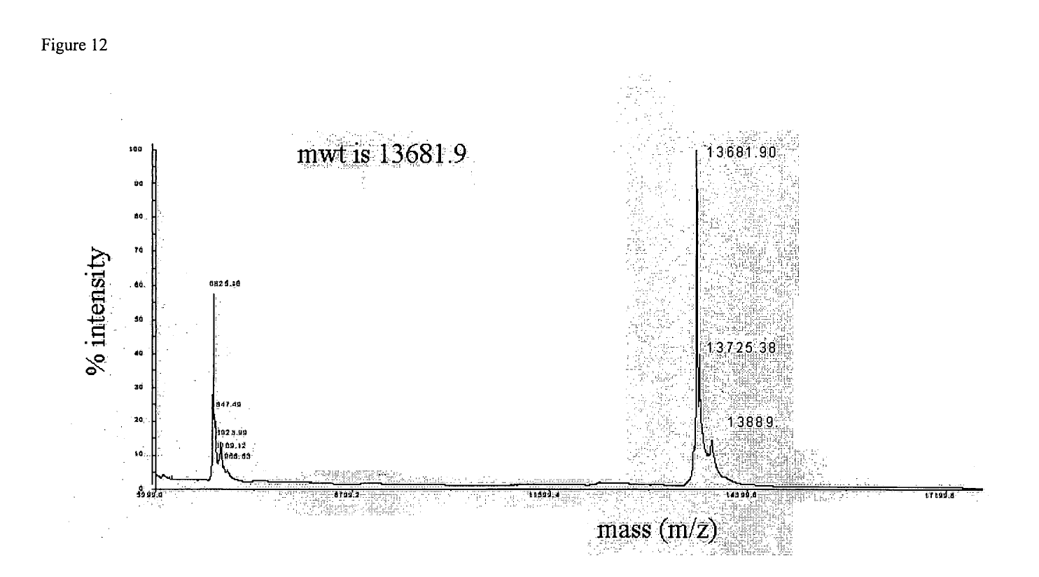

The molecular mass of TrkCIg₂6His was determined using a PE Biosystems voyager-DE STR MALDITOF mass spectrometer, with a nitrogen laser separating at 337 nm. The matrix solution was freshly prepared sinapinic acid at a concentration of 1 mg/100 μl in a 50:50 mixture of acetonitrile at 0.1% trifluoracetic acid. 0.5 μl of sample and matrix were spotted onto the sample plate. The sample was calibrated against Calmix 3 (PE Biosystems) run as a close external standard. The spectrum was acquired over the range 5000-80,000 Da, under linear conditions with an accelerating voltage of 25,000 V, an extraction time of 750 ns and a laser intensity of 2700. Results are shown in FIG. 12.

The molecular weight of TrkCIg₂6His was found to be 13,681.9 Da. This is almost exactly as predicted by a theoretical calculation of the molecular weight (13,685.3 Da) taking into account loss of the N-terminal methionine, which we have previously found to be removed in proteins incorporating the 6 histidine tag from the expression vector pET15b.

Activity Studies: Binding Activity of TrkAIg₂6His, TrkAIg_{2.6}6His, TrkBIg₂6His and TrkCIg₂6His

The resulting monomeric recombinant TrkIg₂ were shown to bind the natural ligands of the respective full length receptors with similar affinity to the wild type receptor i.e. this may be expected to be biologically active. In contrast strand swapped dimeric TrkBIg₂6His would be biologically inactive.

The ability of TrkIg₂ domains to bind to their respective ligands was measured using plasmon surface resonance with a BiaCore system (BiaCore). TrkIg₂ was bound to the matrix of a CM5 chip by amine coupling.

Binding Activity of TrkIgs. Surface Plasmon Resonance

i. TrkBIg₂₆His

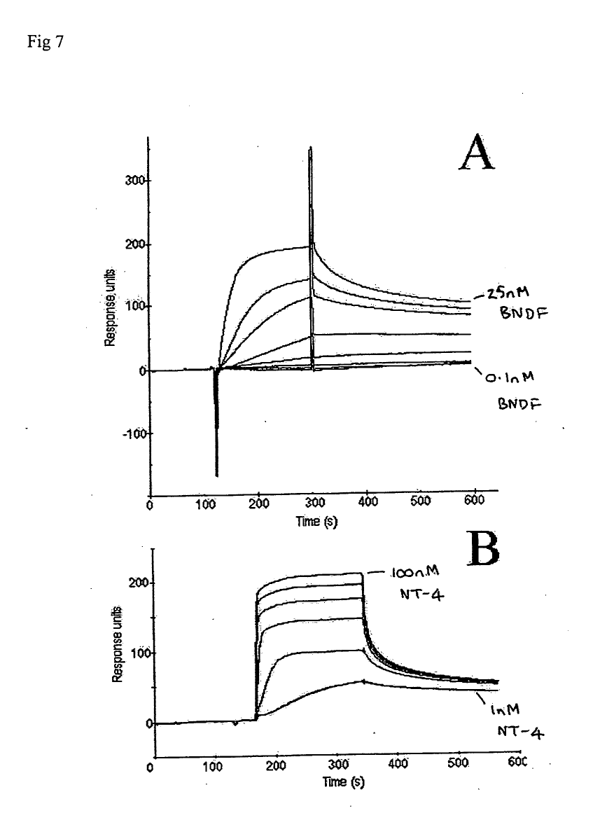

BDNF was passed over the chip at 0.1-25 nM. Association and dissociation rates were estimated according to a 1:1 Langmuir binding model, giving a KD of 790 pM. NT-4 was passed over the chip at 1-100 nM. Association and dissociation rates were estimated according to a 1:1 Langmuir binding model, giving a KD of 260 pM. Results are shown in FIG. 7. FIG. 7A shows the results of experiments with BDNF at 0.1, 0.2, 0.5, 1, 2, 5, 10 and 25 nM (all duplicate). Association and dissociation were fitted to a 1:1 Langmuir model, giving a KD of 790 pM (Chi²=4.39).

FIG. 7B shows the results of experiments with NT-4 at 1, 5, 25, 50, 75 and 100 nM (all duplicate). Association and dissociation were fitted to a 1:1 Langmuir model, giving a KD of 260 pM (Chi²=2.85).

ii. TrkAIg₂6His

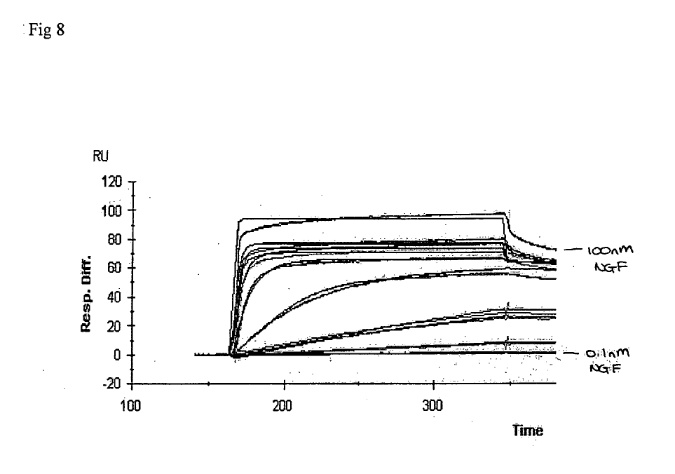

NGF was passed over the chip at 0.1-100 nM. Association and dissociation rates were estimated according to a 1:1 Langmuir binding model, giving a KD of 92.6 pM. The results are shown in FIG. 8.

iii. TrkAIg_{2.6}6His

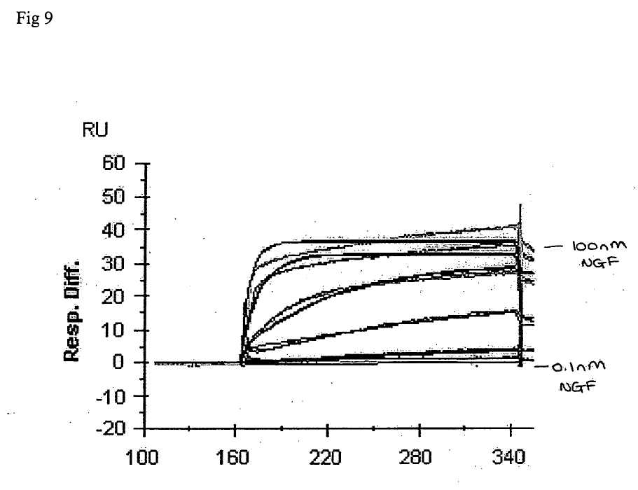

NGF was passed over the chip at 0.1-100 nM. Association and dissociation rates were estimated according to a 1:1 Langmuir binding model, giving a KD of 79.2 pM. Results are shown in FIG. 9. This is a very high affinity and commensurate with known characteristics of the biological membrane bound wild type receptor.

Djouhri, L. et al (supra) indicates TrkAIg₂6His is active in vivo to prevent abnormal fibre firing of noiceptive neurons.

iv. TrkCIg₂6His

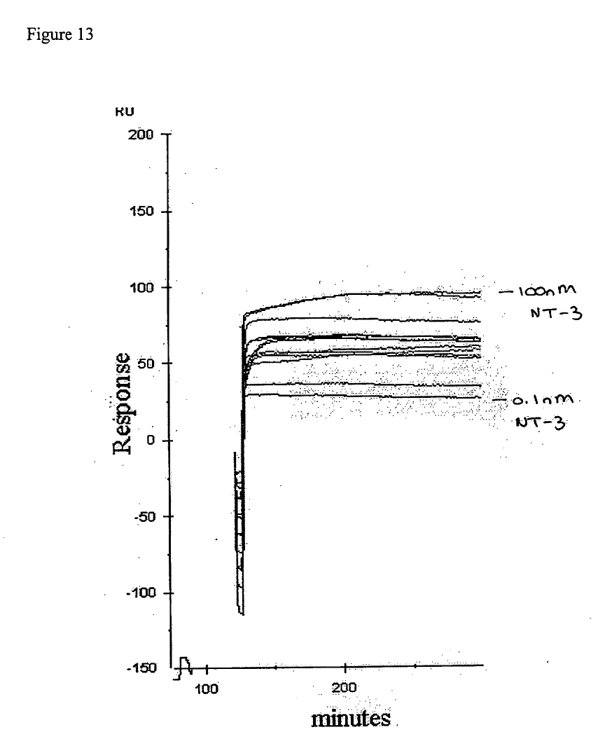

NT-3 was passed over the chip at 0.1-100 nM. Regeneration was with 10 μl 10 mM glycine, pH 1.5. Association and dissociation rates were estimated according to a 1:1 Langmuir binding model, giving KD of 200 μm. The results are shown in FIG. 13.

Production of Non-Histidine-Tagged TrkAIgs

Cloning of TrkAIg₂ noHis

TrkAIg₂ was cloned into pET24a for the expression of TrkAIg₂ without the histidine tag (TrkAIg₂ noHis). Without modification this does not express protein.

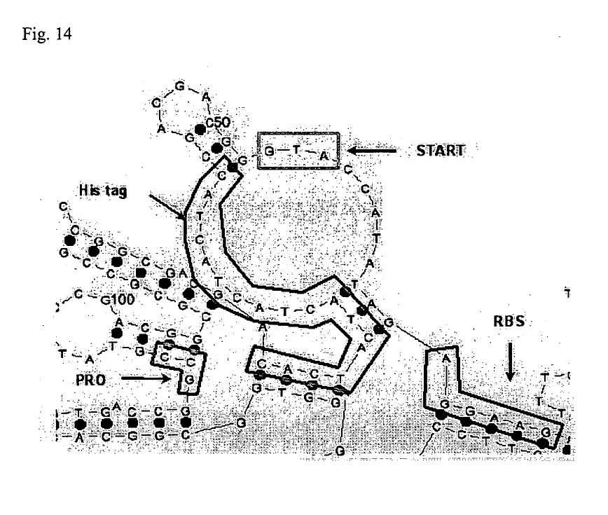

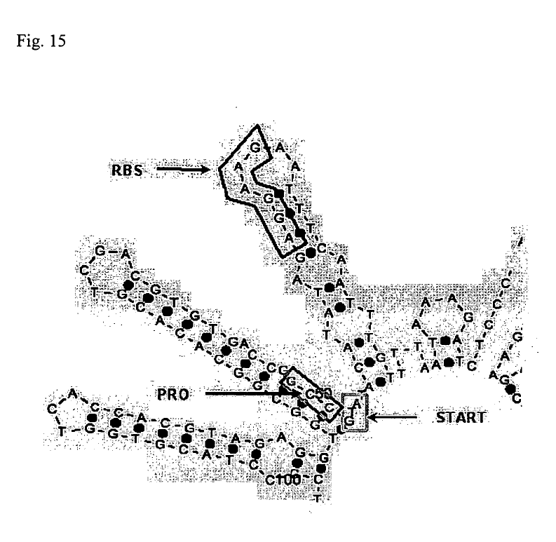

It is known that secondary structure in the mRNA transcript can interfere with the AUG translation initiation codon and/or the ribosome binding site. Using the software MFOLD (http://bioweb.pasteur.fr/seqanal/interfaces/mfold.html) to investigate the secondary structure it was seen that the transcription start site was not ideal for expression. FIG. 14 shows the predicted mRNA structure for TrkAIg₂6His in pET15b. The mRNA coding for the 6His tag is outlined, as is the ribosome binding site (RBS) and the codon for a proline residue (PRO). FIG. 15 shows the predicted mRNA structure of TrkAIg₂ noHis in pET24a. It can be seen that the initiation site is much less accessible than in the 6His version. Similar restrictions also arise in predicted structures of TrkBIg₂noHis and TrkCIg₂noHis.

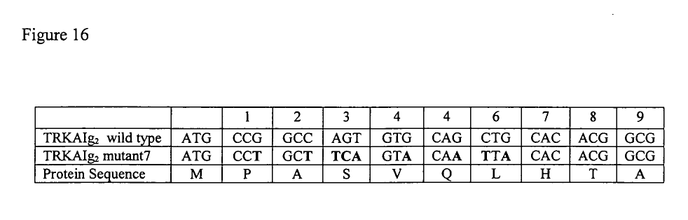

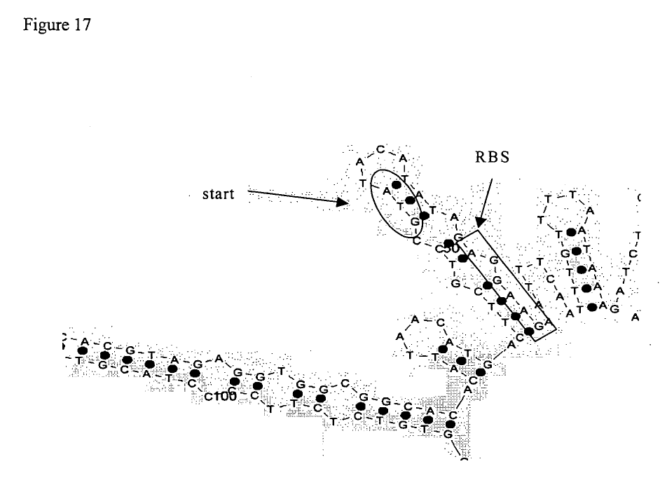

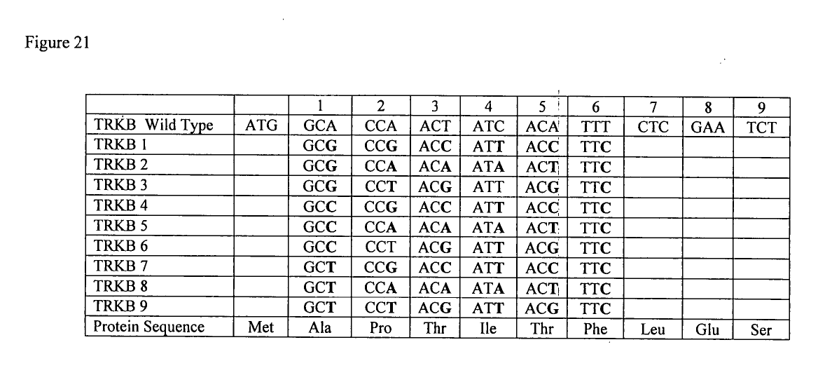

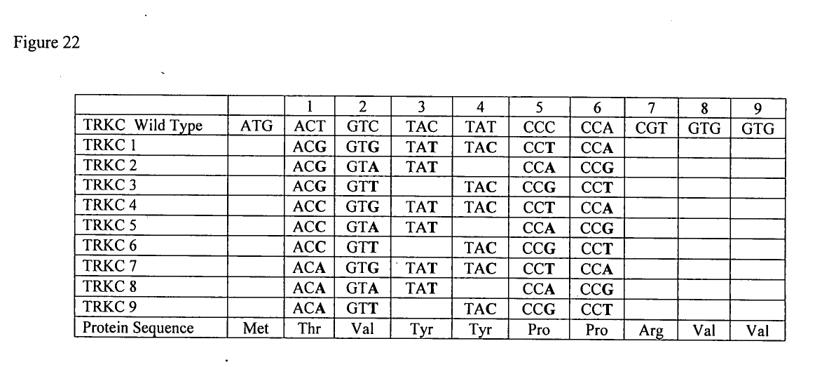

Using computer software to predict the resulting mRNA structures, various silent mutations were introduced into the DNA structure of TrkAIg₂ noHis to allow access to the RBS. FIG. 16 shows an example of a resulting DNA sequence, compared with the wild-type. Mutated bases are marked bold. The resulting mRNA-structure predicted by MFOLD is shown in FIG. 17. Examples of suitable mutated sequences for TrkBIg₂noHis are shown in FIG. 21 and for TrkCIg₂noHis in FIG. 22.

TrkAIg₂ was amplified by PCR from the pET15b-TrkAIg₂6His plasmid using the forward primer GGAATTCCATATGCCTGCTTCAGTACAATTACACACGGCGGTC which incorporates mutated bases and reverse primer CCGCTCGAGTTATCATTCGTCCTTCTTCTCCACCGGGTCTCCA. Primers include sites for NdeI and XhoI respectively at the 5′ and 3′ of TrkAIg₂noHis. Between 100-1000 pmol primers were used per reaction.

Hot start PCR was carried out over 30 cycles in a thermal cycler. After an initial denaturing temperature of 94° C. for 15 minutes, PFU polymerase was added and 30 cycles of denaturation at 94° C. for 1 minute, annealing at 67° C. for 1 minute and extension at 72° C. for 1 minute were carried out. Final extension was 10 minutes at 72° C. followed by a 4° C. holding step. PCR products were analysed by agarose gel electrophoresis TrkAIg₂noHis mutants were subcloned into NdeI and XhoI digested pET24a to create the expression vector pET24a-TrkAIg₂ noHis.

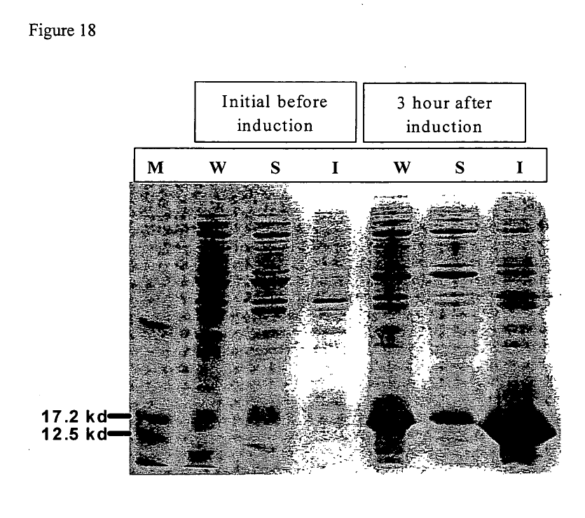

FIG. 18 shows SDS-PAGE analysis of TrkAIg₂ noHis expressed in E. coli; (M) markers, (W) whole cell extract, (S) soluble extract, (1) insoluble extract. It can be seen that TrkAIg₂noHis is expressed mainly in the insoluble fraction.

Production of Recombinant TrkAIg₂ noHis Polypeptide

Electrocompetent E. coli BL21 (DE3) cells were transformed with pET24a-TrkAIg₂ noHis and expression was carried out in accordance with the pET (Novagen) manual. After transformation E. coli lysates were analysed by SDS-PAGE for expression of the 13.5 kDa protein. TrkAIg₂ noHis protein was expressed at high levels in the urea-soluble fraction but not in other fractions. 2 ml of 2YT broth (containing 50 μg/ml kanomycin), was inoculated with a colony which was grown at 37° C. to mid log phase. This was used to inoculate 50 ml of 2YT broth (containing 50 μg/ml kanomycin), which was grown at 37° C. to mid log phase. This was used to inoculate 5′ litres of 2YT broth (containing 50 μg/ml kanomycin) which was grown to an optical density of 1.0 at 600 nm. 1 mM IPTG was added to induce protein expression and cells were grown for 3 hours at 37° C. The harvested cells were resuspended in 10% glycerol and frozen at −80° C. (8 pellets).

Inclusion Body Preparation

Pressed cells were mixed in 20 mM Tris buffer pH 8.5 1 mM PMSF, 10 mM EDTA, gently pipetted and 20 mM Tris buffer pH 8.5 added. These were centrifuged at 9000 rpm for 60 minutes, and supernatant removed. The procedure was repeated with 20 mM Tris buffer pH 8.5, 1 mM PMSF, 10 mM EDTA and 1M NaCl, added and then with 1% Triton X-100 added. Then a final wash was carried out with 20 mM Tris buffer pH 8.5, 1 mM PMSF, 10 mM EDTA. This was subsequently centrifuged at 9000 rpm for 30 minutes. Supernatant was removed. All washes were at 4° C. Inclusion bodies were frozen at −70° C.

Inclusion bodies were solubilised in 8M urea in 20 mM Tris buffer pH 8.5 with 25 mM DTT added for three hours at 14° C.

Refolding of TrkAIg₂ noHis Polypeptide

Insoluble TrkAIg₂ noHis protein contained in the inclusion bodies was released from the cells with an Xpress, and washed with salt and Triton X100 to remove soluble matter. The purified inclusion bodies were solubilised in 8M urea buffer (20 mM Tris pH 8.5, 25 mM DTT) and incubated at room temperature for 3 hours with gentle shaking.

Purification was carried out using an anion exchange column, such as Q Sepharose Fast Flow (Pharmacia), equilibrated and run in 8M urea (pH 8.5) with 10 mM DTT added. Protein was eluted with a gradient of NaCl, in which the protein eluted at approximately 180 mM NaCl or a step at 200 mM NaCl. Eluted protein was refolded at 1 mg/ml on a gel filtration column in Tris pH 8.5 with 100 mM NaCl.

Refolding with gel filtration was successful with a variety of gel filtration media: SuperDex 200, SuperDex 75, Sephacryl HR100, and Sephacryl HR200. In this system, TrkAIg₂ noHis ran with a retention coefficient of 0.55. Unexpectedly it ran a little faster than anticipated compared with TrkAIg₂6His under the same conditions. Increased monomeric form was observed with extended solubilisation. Additionally, the monomeric peak may be finally put onto a Poros Q column to concentrate the protein concentration.

Characterisation of TrkAIg₂ noHis Produced by Method of the Invention

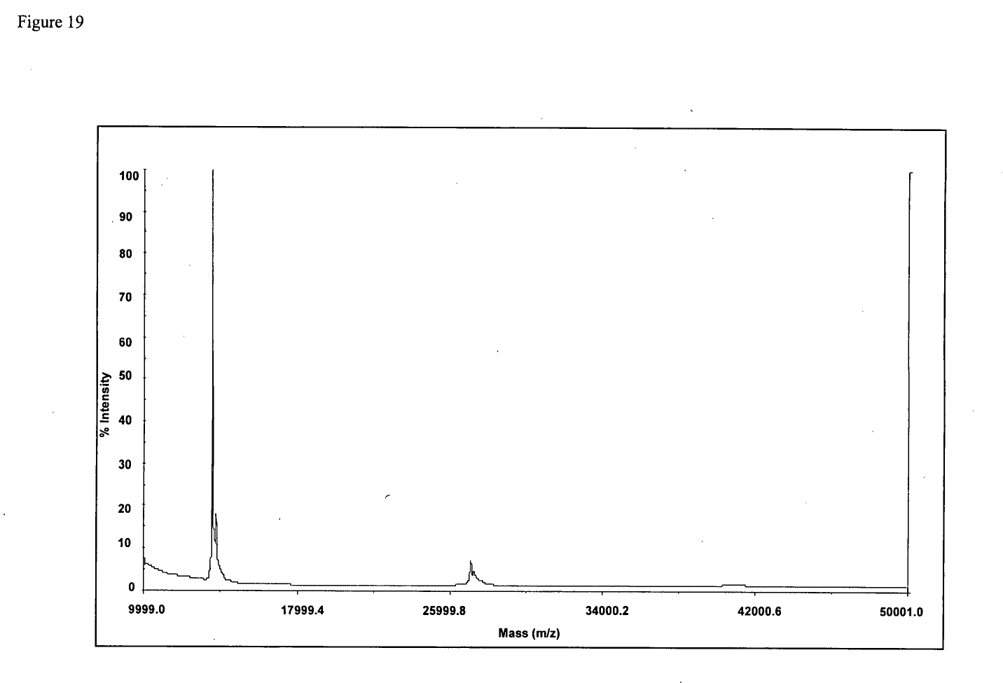

The molecular mass of TrkAIg₂ noHis was determined using a PE Biosystems Voyager-DE STR MALDITOF mass spectrometer with a nitrogen laser operating at 337 nm. The matrix solution was freshly prepared sinapinic acid at a concentration of 100 mg/100 μl in a 50:50 mixture of acetonitrile and 0.1% trifluoracetic acid. 0.5 μl of the sample and matrix were spotted onto the sample plate. The sample was calibrated against Calmix 3 (PE Biosystems) run as a close external standard. The spectrum was acquired over the range 5000-80,000 Da, under linear conditions with an accelerating voltage of 25,000V, an extraction time of 750 nsecs and laser intensity of 2700. Results are shown in FIG. 19.

The molecular weight of TrkAIg₂ noHis was found to be 13,561.2 Da. This is almost exactly as predicted by theoretical calculation of the molecular weight (13,553 Da).

Biological activity of TrkAIg₂ noHis produced by the method of the invention: PC12 cell bioassays

Biological activity of TrkAIg₂ noHis produced by the method of the invention was tested by PC12 neurite outgrowth bioassay. PC12 cells were plated out at 2×10⁴ cells per well in complete DMEM medium (including 100 units/ml penicillin, 100 μg/ml streptomycin, 10% horse serum, 10% FCS and 2 mM glutamine) on collagen-coated 24-well plates. NGF was added at 1 ng/ml and TrkAIg₂ noHis was added at varying concentrations.

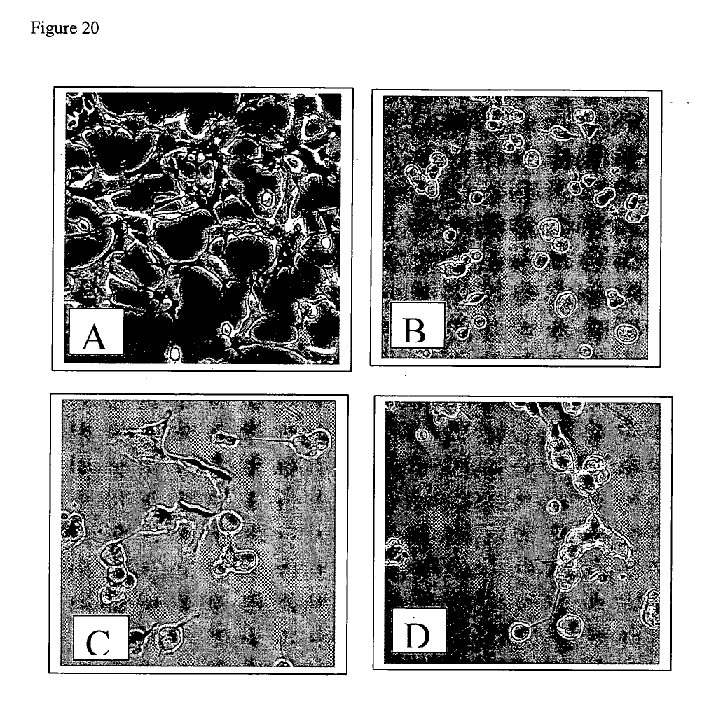

Results from an experiment using TrkAIg₂ noHis refolded on a SuperDex 200 column are shown in FIG. 20. Photographs show neurite outgrowth after 48 hours. Cells were fixed before photographing. FIG. 20A shows neurite outgrowth with 1 ng/ml NGF; FIG. 20B shows no neurite outgrowth when no NGF is added; FIG. 20C shows reduced neurite outgrowth when 2.5 μm TrkAIg₂ noHis is added; FIG. 20D shows no neurite outgrowth when 4.5 μm TrkAIg₂ noHis is added.

Similar results were obtained using TrkAIg₂ noHis refolded on SuperDex 75, Sephacryl HR100 and Sephacryl HR200 columns.

TrkAIg₂noHis was therefore able to prevent neurite growth in response to NGF in the PC12 cell line.

1. A method of producing tyrosine kinase receptor-related polypeptides, the method comprising expressing a tyrosine kinase receptor-related polypeptide in a recombinant expression system and separating expressed monomeric tyrosine kinase receptor-related polypeptide from multimeric form(s) of the expressed polypeptide in a separation step, the separation step allowing refolding of the expressed tyrosine kinase receptor-related polypeptide into a biologically active form.

2. A method according to claim 1 in which the tyrosine kinase receptor is a native TrkA, TrkB, or TrkC; or a biologically active homologue, variant, portion of those receptors or a construct including a homologue, variant, or portion thereof.

3. A method according to claim 2 in which a portion of a tyrosine kinase receptor, or a construct including such a portion, is expressed and in which the portion is selected from the 1 g2 domains of the TrkA, TrkB, and TrkC receptors respectively.

4. A method according to claim 3 in which the polypeptide is selected from TrkAIg₂, TrkAIg_{2.6}, TrkBIg₂ and TrkCIg₂.

5. A method according to claim 4 in which the polypeptide is TrkAIg₂ or TrkAIg_{2.6}

6. A method according to claim 1, 2, 3, 4 or 5 in which the polypeptide is expressed with a histidine tag sequence.

7. A method according to any one of claims 1 to 5 in which the polypeptide is expressed without a histidine tag sequence.

8. A method according to any one of claims 1 to 7 in which the tyrosine kinase sequence is human.

9. A method according to any preceding claim in which the tyrosine kinase receptor-related polypeptide is expressed in insoluble form.

10. A method according to claim 9 in which the tyrosine kinase receptor-related polypeptide is expressed in bacterial inclusion bodies.

11. A method according to any preceding claim in which the multimeric forms include dimers.

12. A method according to any preceding claim in which the polypeptide is able to bind a ligand of the corresponding native tyrosine kinase receptor with high affinity.

13. A method according to any preceding claim in which the separation step involves gel filtration.

14. A method according to any preceding claim in which the separation step is carried out at a salt concentration between and including 0 mM and 500 mM.

15. A method according to any preceding claim in which the separation step is carried out at a salt concentration above 25 mM and below 200 mM.

16. A method according to claim 15 in which the separation step is carried out at a salt concentration of about 100 mM.

17. A method according to any one of claims 13 to 16 in which the gel used in the gel filtration step is able to separate molecules having a molecular weight of about 12 to 40 kDa.

18. A method according to claim 17 in which the gel is Sephadex 200 or SuperDex 200.

19. A method according to claim 18 in which the gel is SuperDex 200.

20. A method according to any preceding claim in which the separation step is carried out at a pH of between 8 and 9.

21. A method according to claim 20 in which the separation step is carried out at a pH of about 8.5.

22. A method according to any preceding claim in which the polypeptide is produced in bacterial-based expression system.

23. A method of purifying recombinant TrkAIg₂ or TrkAIg_{2.6} from inclusion bodies in a bacterial expression system in which monomeric TrkAIg₂ or TrkAIg_{2.6} is separated from a mixture including monomeric and multimeric TrkAIg₂ or TrkAIg_{2.6} by a gel filtration step and allowed to refold into an active form.

24. A method of purifying recombinant TrkBIg₂ from inclusion bodies in a bacterial expression system in which monomeric TrkBIg₂ is separated from a mixture including monomeric and multimeric TrkBIg₂ by a gel filtration step and allowed to refold into an active form.

25. A method of purifying recombinant TrkCIg₂ from inclusion bodies in a bacterial expression system in which monomeric TrkCIg₂ is separated from a mixture including monomeric and multimeric TrkCIg₂ by a gel filtration step and allowed to refold into an active form.

26. A preparation of TrkAIg₂ obtained by a method according to any one of claims 3 to 23 and comprising less than 20% TrkAIg₂ dimer or dimer aggregate.

27. A preparation of TrkAIg₂ according to claim 26 comprising less than 10% TrkAIg₂ dimer or dimer aggregate.

28. A preparation of TrkAIg₂ according to claim 27 comprising less than 1% TrkAIg₂ dimer or dimer aggregate.

29. A preparation of TrkAIg₂ according to claim 28 comprising less than 0.1% TrkAIg₂ dimer or dimer aggregate.

30. A preparation of TrkAIg₂ obtained by a method according to any one of claims 3 to 23 and comprising more than 80% TrkAIg₂ monomer.

31. A preparation of TrkAIg₂ obtained by a method according to any one of claims 3 to 23 and comprising more than 90% TrkAIg₂ monomer.

32. A preparation of TrkAIg₂ obtained by a method according to any one of claims 3 to 23 and comprising more than 99% TrkAIg₂ monomer.

33. A preparation of TrkAIg₂ obtained by a method according to any one of claims 3 to 23 and comprising 100% TrkAIg₂ monomer.

34. A preparation of TrkAIg_{2.6} obtained by a method according to any one of claims 3 to 23 and comprising less than 20% TrkAIg_{2.6} dimer or dimer aggregate.

35. A preparation of TrkAIg_{2.6} according to claim 34 comprising less than 10% TrkAIg_{2.6} dimer or dimer aggregate.

36. A preparation of TrkAIg_{2.6} according to claim 35 comprising less than 1% of TrkAIg_{2.6} dimer or dimer aggregate.

37. A preparation of TrkAIg_{2.6} according to claim 36 comprising less than 0.1% of TrkAIg₂ dimer or dimer aggregate.

38. A preparation of TrkAIg_{2.6} obtained by a method according to any one of claims 4 to 23 and comprising more than 80% TrkAIg_{2.6} monomer.

39. A preparation of TrkAIg_{2.6} obtained by a method according to any one of claims 4 to 23 and comprising more than 90% TrkAIg_{2.6} monomer.

40. A preparation of TrkAIg_{2.6} obtained by a method according to any one of claims 4 to 23 and comprising more than 99% TrkAIg_{2.6} monomer.

41. A preparation of TrkAIg₂ obtained by a method according to any one of claims 4 to 23 and comprising 100% TrkAIg_{2.6} monomer.

42. A preparation of TrkBIg₂ obtained by a method according to any one of claims 3, 4, 6 to 22 and 24, and comprising less than 20% TrkBIg₂ dimer or dimer aggregate.

43. A preparation of TrkBIg₂ according to claim 42 comprising less than 10% TrkBIg₂ dimer or dimer aggregate.

44. A preparation of TrkBIg₂ according to claim 43 comprising less than 1% TrkBIg₂ dimer or dimer aggregate.

45. A preparation of TrkBIg₂ according to claim 44 comprising less than 0.1% of TrkBIg₂ dimer or dimer aggregate.

46. A preparation of TrkBIg₂ obtained by a method according to any one of claim 3, 4, 6, to 22 and 24 and comprising more than 80% TrkBIg₂ monomer.

47. A preparation of TrkBIg₂ obtained by a method according to any one of claims 3, 4, 6 to 22 and 24 and comprising more than 90% TrkBIg₂ monomer.

48. A preparation of TrkBIg₂ obtained by a method according to any one of claims 3, 4, 6 to 22 and 24 and comprising more than 99% TrkBIg₂ monomer.

49. A preparation of TrkBIg₂ obtained by a method according to any one of claims 3, 4, 6 to 22 and 24 and comprising 100% TrkBIg₂ monomer.

50. A preparation of TrkCIg₂ obtained by a method according to any one of claims 3, 4, 6 to 22 and 25 and comprising less than 20% TrkCIg₂ dimer or dimer aggregate.

51. A preparation of TrkCIg₂ according to claim 50 comprising less than 10% TrkCIg₂ dimer or dimer aggregate.

52. A preparation of TrkCIg₂ according to claim 51 comprising less than 1% TrkCIg₂ dimer or dimer aggregate.

53. A preparation of TrkCIg₂ according to claim 52 comprising less than 0.1% TrkCIg₂ dimer or dimer aggregate.

54. A preparation of TrkCIg₂ obtained by a method according to any one of claims 3, 4 6 to 22 and 25 and comprising more than 80% TrkCIg₂ monomer.

55. A preparation of TrkCIg₂ obtained by a method according to any one of claims 3, 4, 6 to 22 and 25 and comprising more than 90% TrkCIg₂ monomer.

56. A preparation of TrkCIg₂ obtained by a method according to any one of claims 3, 4, 6 to 22 and 25 and comprising more than 99% TrkCIg₂ monomer.

57. A preparation of TrkCIg₂ obtained by a method according to any one of claims 3, 4, 6 to 22 and 25 and comprising 100% TrkCIg₂ monomer.
